(19) United States
(12) Patent Application Publication
(10) Pub. No.: US 2026/0041823 A1
(43) Pub. Date: Feb. 12, 2026

(54) IMPLANTABLE MATERIAL AND SYNTHESIS METHOD THEREFOR AND PROSTHETIC HEART VALVE

(71) Applicant: VENUS MEDTECH (HANGZHOU) INC., Hangzhou (CN)

(72) Inventors: Lichao Wang, Hangzhou (CN); Xing Wei, Hangzhou (CN); Dajun Kuang, Hangzhou (CN)

(21) Appl. No.: 19/361,034

(22) Filed: Oct. 17, 2025

Related U.S. Application Data
(63) Continuation of application No. PCT/CN2023/112846, filed on Aug. 14, 2023.

(30) Foreign Application Priority Data
Apr. 17, 2023 (CN) 202310421274.7

(51) Int. Cl. A61L 31/06; A61F 2/24; C08G 18/12

(52) U.S. Cl. CPC A61L 31/06; A61F 2/2412; C08G 18/12; A61L 2430/20

(57) ABSTRACT

The present disclosure discloses an implantable material, a synthesis method therefor, and a prosthetic heart valve. The implantable material is made of polyurethane, whose molecular chain structure includes soft and hard segments. Functional side chains are grafted onto the hard segment, and the functional side chains have a solubility parameter in a range of 6 to 8. In the present disclosure, the introduction of functional side chains into the hard segments enhances compatibility between the hard and soft segments while increasing stress transfer points between them. This results in implantable polymer material with superior mechanical properties.

### CROSS-REFERENCE TO RELATED APPLICATIONS

The present disclosure is a Continuation Application of PCT Application No. PCT/CN2023/112846, filed on Aug. 14, 2023, which claims priority to Chinese Patent Application No. 202310421274.7, filed on Apr. 17, 2023, the entire contents of which are hereby incorporated by reference.

### TECHNICAL FIELD

The present disclosure relates to the technical field of prosthetic valves, and in particular to an implantable material and a synthesis method therefor, and a prosthetic heart valve.

### BACKGROUND

Implantable polymer materials refer to polymer materials used to make artificial prostheses that come into contact with the human biological environment, such as polyurethane (PU) materials.

### Technical Problems

To enhance the biocompatibility and biostability of polyurethane materials, polydimethylsiloxane diol is introduced as one of the soft segment raw materials. However, the incorporation of polydimethylsiloxane (PDMS) significantly reduces the mechanical properties of polyurethane materials. This is mainly due to the large difference in solubility parameters between the soft segment (PDMS) and the hard segment (PU), which leads to a distinct interface therebetween. This interface hinders the transmission of stress from the soft segment to the hard segment, causing stress to accumulate at the interface, thereby affecting the mechanical properties of the PDMS-PU material, which is manifested as a significant decrease in tensile strength at the same elastic modulus.

### SUMMARY

In view of the existing technical problems, the present disclosure provides a modified implantable material that enhances the mechanical properties of the material while ensuring biocompatibility and biostability.

The implantable material of the present disclosure is made of polyurethane, wherein the polyurethane has a molecular chain structure including soft and hard segments. The hard segments are grafted with functional side chains, which have a solubility parameter in a range of 6 to 8.

Optionally, the functional side chains include at least one of the following: a dimethylsiloxane group, a polydimethylsiloxane group (PDMS), a phosphorylcholine group, and a sulfonic acid group.

Optionally, the soft segment raw material is an oligomer diol. More preferably, the soft segment raw material is an oligomer diol, such as at least one of the following: a polycarbonate diol, a polyester diol, a polyether diol, and a polydimethylsiloxane diol.

Optionally, the polycarbonate diol is at least one of DURANOL T5651 and DURANOL T5652.

Optionally, the polyether diol is at least one of polyhexanediol (PHMO) and polytetrahydrofuran diol (PTMO).

Optionally, the polyester diol is at least one of adipic acid polyester diol and succinic acid polyester diol.

Optionally, the soft segment includes a first soft segment and a second soft segment.

Optionally, the first soft segment raw material has a molecular weight in a range of 500 to 2000 and a solubility parameter in a range of 6 to 7.

Optionally, the second soft segment raw material has a molecular weight in a range of 500 to 2000, and a solubility parameter in a range of 8 to 10, such as 8.5 to 9.5.

Optionally, the mass ratio of the first soft segment to the second soft segment in the polyurethane is in a range of 2.5 to 5:1.

Optionally, the hard segment includes:
a first main chain unit derived from isocyanate; and
a second main chain unit derived from a chain extender.

The functional side chain is grafted to at least one of the first main chain unit and the second main chain unit.

Optionally, the first main chain unit raw material is selected from at least one of the following: toluene diisocyanate (TDI), hexamethylene diisocyanate (HDI), diphenylmethane diisocyanate (MDI), naphthalene diisocyanate (NDI), paraphenylene diisocyanate (PPDI), isophorone diisocyanate (IPDI), xylylene diisocyanate (XDI), and triphenylmethane triisocyanate.

Optionally, the second main chain unit raw material is selected from at least one of a small molecule diol and a small molecule diamine.

The small molecule diol is selected from at least one of ethylene glycol, butanediol (BDO), hexanediol, and octanediol;

The small molecule diamine is selected from ethylenediamine, butanediamine, and hexamethylenediamine. Optionally, the functional side chain has a molecular weight in a range of 100 to 2000.

Optionally, the functional side chains may be grafted in any of the following three ways:
onto the first main chain unit only;
onto the second main chain unit only; and
onto both the first main chain unit and the second main chain unit. Optionally, when the functional side chains are only grafted onto the first main chain unit, the first main chain unit grafted with the functional side chain accounts for 5% to 20% of the total molar amount of all first main chain units.

Optionally, when the functional side chains are only grafted onto the second main chain units, the second main chain units grafted with functional side chains account for 5% to 20% of the total molar amount of all second main chain units.

Optionally, when the functional side chains are grafted onto both the first and second main chain units, the first and second main chain units grafted with the functional side chains account for 5% to 20% of the total molar amount of all the first and second main chain units.

Optionally, the hard segments (including functional side chains) account for 35% to 55% of the total mass of the polyurethane.

Optionally, the polyurethane has an isocyanate index R value in the range of 1.0 to 1.1.

Optionally, the hard segments have a solubility parameter in the range of 11 to 14.

The present disclosure further provides a method for synthesizing an implantable material, including: providing raw materials for soft and hard segments, and reacting the raw materials to generate the implantable material.

The hard segment raw materials include isocyanate and a chain extender, wherein at least one of the isocyanate and the chain extender is grafted with a functional side chain, and the functional side chain has a solubility parameter in a range of 6 to 8.

Optionally, the functional side chain is selected from at least one of the following: a dimethylsiloxane group, a polydimethylsiloxane group (PDMS), a phosphorylcholine group, and a sulfonic acid group.

Optionally, the functional side chain has a molecular weight in a range of 100 to 2000.

Optionally, the functional side chain is pre-grafted onto isocyanate and/or the chain extender serving as the hard segment raw material.

Optionally, the functional side chain is grafted during the reaction process.

Optionally, the synthesis method includes mixing all raw materials and reacting them to generate the implantable material.

Optionally, the reaction temperature is in a range of 60° C. to 80° C. for a duration of 2 hours to 6 hours.

Optionally, the synthesis method includes:
S100, pre-polymerizing and end-capping the soft segment raw material using isocyanate to obtain a prepolymer; and
S200, reacting the prepolymer with a chain extender to obtain the implantable material.

Optionally, the isocyanate in step S100 is a first isocyanate, and a second isocyanate is further added in step S200, wherein the first isocyanate and the second isocyanate have identical or different structures.

Optionally, the first isocyanate and the second isocyanate are each a single isocyanate or a mixture of multiple isocyanates.

Optionally, the mixture of the multiple isocyanates has a composition selected from:
a. isocyanates all free of grafted functional side chains;
b. isocyanates all grafted with the functional side chains; or
c. a combination of isocyanates with and without the grafted functional side chains.

Optionally, the molar ratio of the isocyanates free of grafted functional side chains to the isocyanates grafted with the functional side chains in the mixture of the multiple isocyanates is in a range of 4:1 to 20:1.

Optionally, the isocyanate free of grafted functional side chains is selected from at least one of the following: toluene diisocyanate (TDI), hexamethylene diisocyanate (HDI), diphenylmethane diisocyanate (MDI), naphthalene diisocyanate (NDI), p-phenylene diisocyanate (PPDI), isophorone diisocyanate (IPDI), xylylene diisocyanate (XDI), and triphenylmethane triisocyanate.

Optionally, the soft segment raw material in step S100 includes a first soft segment raw material and a second soft segment raw material, and both the first soft segment raw material and the second soft segment raw material are pre-polymerized and end-capped with isocyanate.

Optionally, the first soft segment raw material has a molecular weight in a range of 500 to 2000 and a solubility parameter in a range of 6 to 7.

Optionally, the second soft segment raw material has a molecular weight in a range of 500 to 2000 and a solubility parameter in a range of 8 to 10, such as 8.5 to 9.5.

Optionally, the first soft segment raw material is polydimethylsiloxane diol.

Optionally, the second soft segment raw material is selected from at least one of the following: polyether diol, polycarbonate diol, and polyester diol, for example, at least one of the following: polyhexanediol (PHMO), polytetrahydrofuran diol (PTMO), Duranol T5651, and Duranol T5652.

Optionally, the mass ratio of the first soft segment raw material to the second soft segment raw material is in a range of 2.5:1 to 5:1.

Optionally, in step S100, the first soft segment raw material and the second soft segment raw material are pre-mixed prior to feeding.

Optionally, the chain extender includes a linear chain extender and a chain extender grafted with functional side chains.

Optionally, the linear chain extender is at least one of a small molecule diol and a small molecule diamine, such as at least one of the following: ethylene glycol, propylene glycol, butanediol, hexanediol, octanediol, and ethylenediamine.

Optionally, the main chain of the chain extender grafted with functional side chains is identical to that of the linear chain extender. The chain extender grafted with functional side chains is at least one of 1-palmitoylpropanediol-3-phosphocholine, 9-(2-hydroxy-1-hydroxymethyl-1-methyl-ethylcarbamoyl)-nonylphosphatidylcholine, and sphingosine phosphocholine.

Optionally, the molar ratio of the linear chain extender to the chain extender grafted with functional side chains is in a range of 4:1 to 20:1.

Optionally, a mixture of a linear chain extender and a chain extender grafted with functional side chains is added in step S200.

Optionally, step S200 includes:
adding a chain extender grafted with the functional side chain to react with the prepolymer in a first reaction stage; and
adding a linear chain extender after completion of the first reaction stage and continuing the reaction in a second reaction stage.

Optionally, the temperature of the first reaction stage is in a range of 60° C. to 80° C., with a duration in a range of 1 hour to 2 hours.

Optionally, the temperature of the second reaction stage is in a range of 60° C. to 80° C., with a duration in a range of 1 hour to 2 hours.

Optionally, the chain extension reaction system further includes a solvent, and the solvent is selected from at least one of the following: N,N-dimethylacetamide (DMAc), N,N-dimethylformamide (DMF), tetrahydrofuran (THF), dimethyl sulfoxide (DMSO), and toluene; preferably methylacetamide (DMAc), dimethylformamide (DMF) or tetrahydrofuran (THF).

Optionally, the pre-polymerization temperature in step S100 is in a range of 60° C. to 80° C., with a duration in a range of 2 hours to 4 hours.

Optionally, the pre-sealing temperature in step S100 is in a range of 60° C. to 80° C., with a duration in a range of 2 hours to 4 hours.

Optionally, the reaction temperature in step S200 is in a range of 60° C. to 80° C., with a duration in a range of 2 hours to 6 hours.

The present disclosure further provides a prosthetic heart valve, including a stent and valve leaflets connected to the stent, wherein the leaflets are made of the implantable material, and the valve leaflets include multiple pieces that cooperate with each other to control the opening and closing of the blood flow passage.

The implantable material may be fabricated into valve leaflets using existing processes. Additionally, the valve leaflets may undergo annealing treatment under conditions of 60° C. to 150° C. for 2 hours to 60 hours.

Optionally, the prosthetic heart valve further includes a covering membrane connected to the inner side and/or outer side of the stent, and the covering membrane is made of the implantable material.

### Beneficial Effects

Compared with the prior art, the present disclosure introduces functional side chains into the hard segments. Due to the large steric hindrance of the functional side chains, while ensuring sufficient chain mobility, the side chains will be preferentially arranged in the outer layer of the hard segments without affecting the interaction between the hard segments. Meanwhile, the side chains exhibit better compatibility with the soft segments compared with the hard segments. One end of the side chain forms a chemical bond with the hard segments, while the other end exhibits similar compatibility with the soft segments, effectively bridging the hard and soft segments. This enhances compatibility between the hard and soft segments and increases the number of stress transfer points between them. The stress is well transmitted between the hard and soft segments, preventing stress accumulation at the interface. This results in implantable polymer materials with outstanding mechanical properties.

### BRIEF DESCRIPTION OF DRAWINGS

FIG. 1 is a schematic structural diagram of a polyurethane molecular chain in one embodiment;

FIG. 2 is a schematic diagram of a polyurethane system in one embodiment;

FIG. 3 is a flow chart of a synthesis method for an implantable material according to an embodiment;

FIG. 4 is a schematic diagram showing the synthesis mechanism of an implantable material according to an embodiment;

FIG. 5 is a flow chart of a synthesis method for an implantable material in another embodiment.

### DESCRIPTION OF EMBODIMENTS

Technical solutions of embodiments of the present disclosure will be clearly and completely described below in combination with the drawings. Obviously, the described embodiments are only part of, rather than all of the embodiments of the present disclosure. Based on the embodiments of this disclosure, all other embodiments obtained by those ordinary skilled in the art without any creative work shall fall within the protection scope of this disclosure.

Unless defined otherwise, all technical and scientific terms used herein have the same meaning as commonly understood by one of ordinary skill in the art to which this disclosure belongs. The terms used in the specification of the present disclosure are only for the purpose of describing specific embodiments and are not intended to limit the present disclosure. The term “and/or” as used herein includes any and all combinations of one or more of the associated listed items.

In the field of polymer material synthesis, such as polyurethane material synthesis, the concepts of hard and soft segments in the molecular structure are relative. From a raw material perspective, the hard segment is a structural chain unit formed by low-molecular polyols/amines and isocyanates, and the soft segment is a structural chain unit formed by large-molecular polyols. In terms of chain length, the molecular chains of hard segments are relatively short, with a higher number of functional groups per unit chain length and stronger intermolecular forces, exhibiting rigidity. Conversely, the molecular chains of soft segments are longer, with fewer functional groups per unit chain length and relatively weaker intermolecular forces, exhibiting flexibility.

In the polyurethane system, the soft segment typically forms the continuous phase, while the hard segment serves as the dispersed phase that disperses within the soft segment. Conventional polyurethane materials include polyether polyurethane (PEU), polyester polyurethane, polycarbonate polyurethane (PCU), and polydimethylsiloxane polyurethane (PDMS-PU). These four types of polyurethane materials, whose hard segment raw materials are isocyanate and chain extender, are named primarily according to the soft-segment raw materials. Due to the strong intermolecular forces within the hard segments, they exhibit an ordered, densely packed structure, which is relatively stable. In contrast, the soft segments are the most susceptible to external influences. Among these, the soft segment raw material for PEU is polyether, which is prone to oxidation itself, and the resulting polyurethane also has the problem of easy oxidation. The soft segment raw material for polyester polyurethane is polyester, which is easily hydrolyzed itself, and the polyurethane formed from it also has the problem of easy hydrolysis. The soft segment raw material for PCU is polycarbonate, which is more stable than polyether polyester, but it is still easily hydrolyzed, which consequently imparts hydrolytic instability to the resulting polyurethane. The soft segment raw material for PDMS-PU is polydimethylsiloxane, which has excellent biostability and biocompatibility. As a leaflet material, it offers excellent antioxidant and hydrolysis resistance. However, due to the significant difference between the solubility parameters of polydimethylsiloxane and the hard segment, a distinct interface is formed. Stress cannot be transmitted at the interface and accumulates instead, resulting in poor mechanical properties in the prepared polyurethane.

Current technical approaches for improving the mechanical properties of PDMS-PU can be broadly categorized into the following three types:
(1) Introducing a second soft segment, such as polyether, while allowing some of the hard segments to form an intermediate phase that connects the soft and hard segments, thereby enhancing the compatibility therebetween.
(2) Modifying the groups at both ends of the soft segment, such as attaching carbon chains to both ends of PDMS, and allowing the resulting alkyl end groups and some hard segments to form an intermediate phase, thereby improving the compatibility between PDMS and the hard segment.
(3) Introducing a siloxane group into the (linear) main chain of the hard segment by using a linear siloxane chain extender, such as 1,3-bis(4-hydroxybutyl)tetramethyldisiloxane (BHTD). On one hand, this reduces the polarity of the hard segments and improves their compatibility with PDMS. On the other hand, the incorporation of BHTD disrupts the ordering of the hard segments (due to steric and structural effects), increasing the entropy of the hard segments themselves, reducing phase separation with PDMS, and thus enhancing the compatibility between the hard and soft segments.

Compared with the improvement approaches (1) and (2), the PDMS-PU in approach (3) exhibits superior biostability because the polysiloxane lacks functional groups of polyether, polyester, or polycarbonate, resulting in enhanced stability. However, introducing carbon chains at both ends of the polysiloxane or incorporating a second soft segment represents a compromise in the mechanical properties (a trade-off of performance), yet it still increases the instability factor, failing to meet the disclosure requirements of prosthetic valves.

The inventors of this disclosure have identified that one of the main factors affecting the mechanical properties of polyurethanes is the significant difference in the solubility parameters between the hard and soft segments. Their poor compatibility hinders smooth stress transfer therebetween, causing stress concentration in either the soft or hard segments. This, in turn, compromises the mechanical properties of the polyurethanes, for example, causing them to break or crack easily during application. Therefore, starting from improving the compatibility of solubility parameters, this disclosure introduces suitable functional side chains into the hard segments, enabling the polyurethane to achieve both enhanced mechanical properties and biocompatibility.

An embodiment of the present disclosure provides an implantable material made of polyurethane. The polyurethane molecular chain structure includes hard and soft segments, wherein the hard segments are grafted with functional side chains, and the functional side chain has a solubility parameter of 6 to 8. Specifically, the functional side chains may be selected from at least one of the following: a dimethylsiloxane group, a polydimethylsiloxane group, a phosphorylcholine group, and a sulfonic acid group, such as PDMS, or a phosphorylcholine group.

In this disclosure, the molecular weight of the polyurethane is not strictly limited, generally being above 50,000, for example, 50,000 to 300,000. It can be adjusted based on specific performance requirements and determined in conjunction with the molecular weight ranges of the soft and hard segments described below.

In the present embodiment, functional side chains are introduced into the structural chain of the hard segment. On one hand, the length of the main chain remains unaffected, thus preventing a decrease in hard segment strength. On the other hand, guided by the principle of lowest-energy arrangement, the hard segment tends to form internally ordered segments, while the functional side chains tend to localize at the periphery of these segments. Structurally, one end of the functional side chain is chemically bonded to the hard segment, and the other end is miscible with the soft segment based on the principle of like dissolves like. This effectively bridges the soft and hard segments and further eliminates interfacial stress therebetween. Meanwhile, due to the low surface energy of the functional side chains, they tend to migrate to the polyurethane surface. This provides enhanced protection to the soft segments while enabling the polyurethane to exhibit the hydrophobicity, biostability, and excellent biocompatibility inherent to the side chains. Consequently, the mechanical properties of the polyurethane material are enhanced. The polyurethane of this embodiment has a molecular structure as follows:

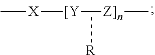

wherein X represents the soft segments, [Y—Z]ₙ represents the hard segments, and n ranges from 1 to 4; and
Y denotes the first main chain unit, Z denotes the second main chain unit, R is a functional side chain, with at least one of Y and Z grafted with R.

In different [Y—Z] units, Y and Z may be identical or different.

The soft segment raw material may be a polymer polyol, for example, an oligomer polyol, such as an oligomer diol, which may be selected from at least one of the following: polycarbonate diol, polyester diol, polyether diol, and polydimethylsiloxane diol, such as polydimethylsiloxane diol with a molecular weight of 500 to 2000. The polycarbonate diol is at least one of DURANOL T5651 and DURANOL T5652. The polyether diol is at least one of polyhexanediol (PHMO) and polytetrahydrofuran diol (PTMO), such as polyhexanediol with a molecular weight of 500 to 2000. The polyester diol is at least one of adipic acid polyester diol and succinic acid polyester diol.

The first main chain unit in the hard segment is formed by the isocyanate, while the second main chain unit is formed by the chain extender. Specifically, the first main chain unit raw material may be selected from at least one of the following: toluene diisocyanate (TDI), hexamethylene diisocyanate (HDI), diphenylmethane diisocyanate (MDI), naphthalene diisocyanate (NDI), p-phenylene diisocyanate (PPDI), isophorone diisocyanate (IPDI), xylylene diisocyanate (XDI), and triphenylmethane triisocyanate, such as MDI or triphenylmethane triisocyanate. The second main chain unit raw material is a small molecule diol selected from at least one of the following: ethylene glycol, butanediol, hexanediol, octanediol, and ethylenediamine, such as ethylene glycol or ethylenediamine.

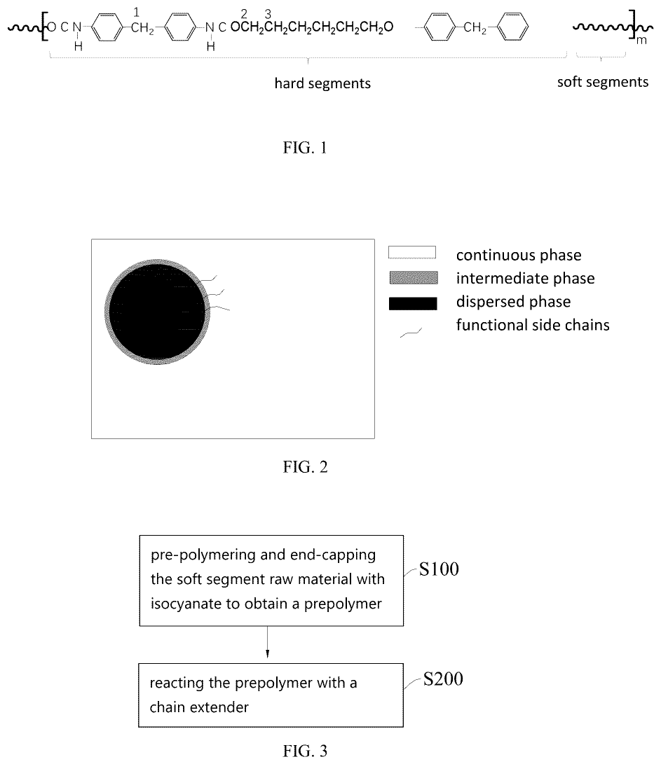

The functional side chain may be grafted onto the first main chain unit only, onto the second main chain unit only, or onto both the first and second main chain units simultaneously. Taking the structure shown in FIG. 1 as an example, the polyurethane main chain is linear, and the functional side chain may be grafted onto site “1” or site “2” or “3” on the hard segment. This, however, is merely an illustrative example, as all short-chain diols (small molecule diols) and isocyanates can serve as optional grafting sites.

The content of functional side chains in the polyurethane molecular structure is one of the key parameters affecting the mechanical properties of implantable materials. An appropriate amount of functional side chains can significantly improve the mechanical properties of implantable materials. When grafted solely onto the first main chain unit, the molar amount of the first main chain unit grafted with the functional side chains accounts for 5% to 20% of the total molar amount of all first main chain units, for example, 10% to 20%, or 12.66%. When grafted solely to the second main chain unit, the molar amount of the second main chain unit grafted with the functional side chains accounts for 5% to 20% of the total molar amount of all second main chain units, for example, 10% to 20%, or 19.23%. When grafted onto both the first and second main chains, the combined molar amount of the first and second main chains grafted with the functional side chains accounts for 5% to 20% of the total molar amount of all first and second main chains, for example, 10% to 15%, or 12.66%. An appropriate amount of functional side chains enhances the compatibility between the hard and soft segments, thereby improving the mechanical properties. However, excessive functional side chains significantly increase steric hindrance within the polyurethane molecular structure, disrupting the ordered arrangement of hard segments. This leads to reduced hard segment stability and consequently degrades the overall mechanical properties of the polyurethane material.

In terms of the functional side chain itself, its molecular weight ranges from 100 to 2000, typically between 500 and 1000. If the molecular weight is too low, the side chain cannot effectively link the hard and soft segments. Conversely, if the molecular weight is too high, the functional side chain will generate significant steric hindrance, making it difficult for the hard segments to be incorporated into the polyurethane chain, and significantly disrupting the order of the hard segments.

As shown in FIG. 2, in the polyurethane system, there is a transitional interface, namely the intermediate phase, that exists between the dispersed and continuous phases, which allows stress transfer between the two phases. Structurally, the intermediate phase is primarily composed of partial hard segments, terminal alkyl groups of soft segments, and functional side chains. The functional side chains, miscible with the soft segments based on the principle of like dissolves like, further facilitate stress transfer between the dispersed and continuous phases, thereby enhancing the mechanical properties of the polyurethane materials.

Based on heat flow analysis, the glass transition temperature (Tg_{med}) of the intermediate phase falls within the range of Tg_{sof} to Tg_{sof}+20° C. of the soft segments. Typically, differential scanning calorimetry (DSC) reveals three distinct Tg curve results, namely those of the dispersed phase, the intermediate phase and the continuous phase. The Tg curve of the intermediate phase exhibits a certain shift towards the Tg curve of the continuous phase, indicating that some hard segments participate in the formation of the intermediate phase.

In another embodiment, the polyurethane has a molecular structure as follows:

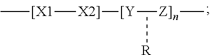

Among them, [X1-X2] represents the soft segment; [Y—Z]ₙ represents the hard segment, and n ranges from 1 to 4; and
Y denotes the first main chain unit, Z denotes the second main chain unit, R is a functional side chain, and at least one of Y and Z is grafted with R;
In different [Y—Z]units, Y and Z may be the same or different.

In this embodiment, the soft segments include first soft segments X1 and second soft segments X2. The first soft segment raw material has a molecular weight of 500 to 2000 and a solubility parameter of 6 to 7. The second soft segment raw material has a molecular weight of 500-2000 and a solubility parameter of 8-10, for example, 8.5 to 9.5. The chemical properties (solubility parameter) of the second soft segments lie between those of the soft and hard segments, mitigating adverse interactions between them, such as poor compatibility and inefficient stress transfer.

In one embodiment, the mass ratio of the first soft segments to the second soft segments in the polyurethane is 2.5 to 5:1, for example, 2.5 to 4:1, or specifically 4:1, which can effectively adjust the solubility parameter of the soft segments, improving the compatibility between the soft and hard segments and thereby enhancing the mechanical properties of the polyurethane.

Based on overall performance considerations for implantable materials, the hard segment (including functional side chains) in the molecular chain structure should constitute 35% to 55% of the total polyurethane molecular weight, such as 40% to 45%, or specifically 45%, to achieve good mechanical strength and elasticity.

The R-value of polyurethane, also known as the isocyanate index, refers to the molar ratio of isocyanate groups (—NCO) to hydroxyl groups (—OH). This R-value significantly affects the performance of polyurethane, primarily affecting its glass transition temperature, swelling degree, and tensile strength. In this disclosure, the R-value of the polyurethane is set between 1.0 and 1.1, for example, 1.0 and 1.05, to achieve the desired performance. Combined with the molecular structural improvements described therein, the resulting material is particularly suitable for applications involving long-term dynamic deformation in vivo, achieving a fatigue life of 200 million cycles.

Regarding the dispersion uniformity of the polyurethane system, the solubility index difference between the soft and hard segments should be controlled within a specific range. For example, the difference between the soft and hard segments should not exceed 8. More preferably, the difference between the soft and hard segments does not exceed 6, with the solubility parameter of the hard segments ranging from 11 to 14.

One embodiment of the present disclosure provides a method for synthesizing an implantable material, including providing a soft segment raw material and a hard segment raw material, and reacting the raw materials to generate the implantable material. The hard segment raw material includes isocyanate and a chain extender, wherein at least one of the isocyanate and the chain extender is grafted with a functional side chain, and the solubility parameter of the functional side chain ranges between 6 and 8.

Specifically, the functional side chain may be at least one of the following: dimethylsiloxane, polydimethylsiloxane group (PDMS), phosphorylcholine, and sulfonic acid groups, such as PDMS or phosphorylcholine. Various combinations of these functional side chains can be grafted with the two hard segment materials in multiple ways. For example, dimethylsiloxane and/or polydimethylsiloxane groups may be grafted onto isocyanate, while phosphorylcholine and/or sulfonic acid groups are grafted onto the chain extender; or dimethylsiloxane and/or polydimethylsiloxane groups may be grafted onto chain extenders, while phosphorylcholine and/or sulfonic acid groups are grafted onto isocyanate.

Regarding the method for introducing the functional side chain into the hard segments, it can be achieved by providing hard segment raw materials that is pre-grafted with the functional side chain. For example, the functional side chains may be pre-grafted onto at least one of the isocyanate or the chain extender. Alternatively, the functional side chains may be grafted onto the segment of the hard segment raw material during the reaction process.

In an exemplary embodiment, isocyanate grafted with polydimethylsiloxane side chains is prepared by reacting polysiloxane monoalcohols/amine with isocyanate, such as reacting 3-((tert-butyldimethylsilyl)oxy)-propanol with triphenylmethane triisocyanate.

In another exemplary embodiment, a chain extender grafted with functional side chains may be prepared by oxidizing a double-bond raw material grafted with the corresponding functional groups to form an epoxy, followed by ring-opening to form a diol. The double-bond raw material grafted with the corresponding functional groups can be at least one of the following: 1,3-divinyl-1,3-dimethyl-1,3-diphenyldisiloxane (CAS: 2627-97-6), vinylpentamethyldisiloxane (CAS: 1438-79-5), and 1-vinyl-1,1,3,3-tetramethyldisiloxane (CAS: 55967-52-7). Alternatively, the double-bond raw material grafted with the corresponding functional groups can also be produced by an elimination reaction of an alcohol or haloalkane.

The chain extender grafted with functional side chains can also be prepared by hydrolyzing a double bond raw material grafted with corresponding functional groups. The double bond raw material grafted with corresponding functional groups may be a diester raw material, such as 1,2-dilauroyl-SN-glycero-3-phosphocholine (CAS: 18194-25-7) and/or 1,2-diacetyl-SN-glycero-3-phosphocholine (CAS: 56782-46-8).

Polyurethane synthesis methods primarily include one-step and two-step methods. The one-step method involves mixing all raw materials or performing a continuous chemical reaction to produce polyurethane, which can then be used as an implantable material. The reaction temperature ranges from 60° C. to 80° C., with the reaction time of 2 hours to 6 hours.

The two-step method, also known as the pre-polymerization method, typically involves reacting oligomer polyols and isocyanates first to generate a low molecular weight prepolymer, and then adding a chain extender to react with the prepolymer to form polyurethane.

Referring to FIG. 3, the present disclosure provides a specific synthesis method of an implantable material based on the pre-polymerization method, including the following steps:

Step S100, pre-polymerizing and end-capping the soft segment raw material with isocyanate to obtain a prepolymer;

Step S200: reacting the prepolymer with a chain extender.

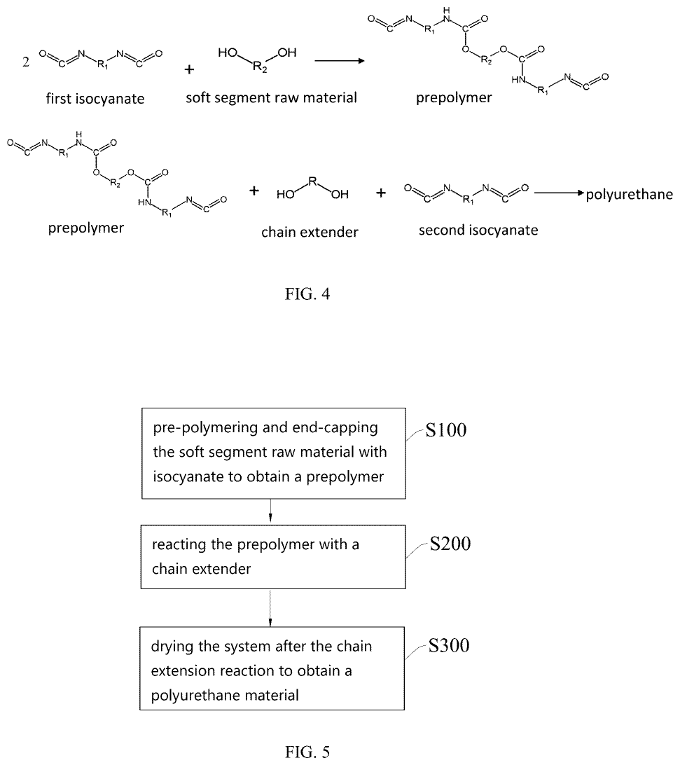

In specific operations, the isocyanate in step S100 may be a first isocyanate, and a second isocyanate may be added in step S200. The specific reaction mechanism is shown in FIG. 4. Here, the first and second isocyanates are collectively referred to as the isocyanates in the hard segment raw materials described above. This terminology is used solely to distinguish between different reaction stages during the synthesis process. Regarding specific types, the first and second isocyanates may be identical or different.

Furthermore, the first isocyanate and the second isocyanate may each be a single isocyanate or a mixture of multiple isocyanates. The mixture of multiple isocyanates may be composed of one of the following: a) isocyanates all without grafted with functional side chains; b) isocyanates all grafted with the functional side chains; c) isocyanates partially grafted with the functional side chains and partially not grafted with the functional side chains.

In a specific embodiment, within the mixture of multiple isocyanates, the molar ratio of the isocyanate without grafted functional side chains to the isocyanate with grafted functional side chains ranges from 4:1 to 20:1, for example 8:1 to 20:1, or specifically 10:1.

In one embodiment, the soft segment raw materials in step S100 include a first soft segment raw material and a second soft segment raw material, both of which are prepolymerized and end-capped with isocyanate. The first soft segment raw material has a molecular weight ranging from 500 to 2000 and a solubility parameter of 6 to 7. The first soft segment raw material may be polydimethylsiloxane diol, such as polydimethylsiloxane diol with a molecular weight of 500 to 2000. The second soft segment raw material has a molecular weight ranging from 500 to 2000 and a solubility parameter of 8 to 10, such as 8.5 to 9.5. The second soft segment raw material may be selected from at least one of polyether diol, polycarbonate diol, and polyester diol. Examples include at least one of the following: polyhexanediol (PHMO), polytetrahydrofuran diol (PTMO), Duranol T5651, and Duranol T5652, such as a polyhexanediol with a molecular weight of 700.

In one embodiment, the mass ratio of the first soft segment raw material to the second soft segment raw material is in the range of 2.5:1 to 5:1, for example 2.5:1 to 4:1, and specially 4:1. The first soft segment raw material and the second soft segment raw material can be pre-mixed before feeding.

In step S200, the chain extender includes a linear chain extender and a chain extender grafted with functional side chains. The linear chain extender is at least one of a small molecule diol and a small molecule diamine, such as ethylene glycol, butanediol, hexanediol, octanediol, and ethylenediamine. The main chain of the chain extender grafted with functional side chains can be the same as that of the linear chain extender. Specifically, the chain extender grafted with functional side chains can be at least one of 1-palmitoylpropanediol-3-phosphocholine (CAS: 68124-68-5), 9-(2-hydroxy-1-hydroxymethyl-1-methyl-ethylcarbamoyl)-nonylphosphatidylcholine, and sphingosine phosphocholine (CAS: 1670-26-4), such as 9-(2-hydroxy-1-hydroxymethyl-1-methyl-ethylcarbamoyl)-nonylphosphatidylcholine. In one embodiment, the molar ratio of the linear chain extender to the chain extender grafted with functional side chains is in the range of 4:1 to 20:1, for example 8:1 to 20:1, and specially 10:1.

In step S200, the chain extender may be introduced by adding a mixture of a linear chain extender and a chain extender grafted with functional side chains, or by adding the chain extender grafted with functional side chains and the linear chain extender sequentially in stages. For example, in one embodiment, step S200 includes a first reaction stage and a second reaction stage. The first reaction stage involves adding the chain extender grafted with functional side chains to react with the prepolymer, and the second reaction stage involves continuing the reaction after adding linear chain extenders following the completion of the first reaction stage. More specifically, the first reaction stage is conducted at 60° C. to 80° C. for 1 to 2 hours, and the second reaction stage is conducted at 60° C. to 80° C. for 1 to 2 hours. This sequential, staged addition method facilitates the participation of the chain extender grafted with functional side chains in polyurethane synthesis.

The chain extension reaction system further includes a solvent, which may be selected from at least one of the following: N,N-dimethylacetamide (DMAc), N,N-dimethylformamide (DMF), tetrahydrofuran (THF), dimethyl sulfoxide (DMSO), and toluene, such as N,N-dimethylacetamide (DMAc). In step S200, the prepolymer and second isocyanate may be fully dissolved in the solvent beforehand or added as needed, to ultimately configure a solution with a concentration of 10 to 30 (w/v) %, for example, 20 (w/v), before the chain extender is added.

During the synthesis process, the pre-polymerization temperature ranges from 60 to 80° C., for example, 80° C., with a duration of 2 to 4 hours. The chain extension reaction temperature ranges from 60 to 80° C., for example, 80° C., with a duration of 2 to 6 hours. Throughout the above synthesis process, an inert gas such as nitrogen is used for both the pre-polymerization end-capping and the chain extension reaction.

Referring to FIG. 5, in one embodiment, the synthesis method further includes:

Step S300: drying the system after the chain extension reaction to obtain an implantable material.

Drying is performed to volatilize the solvent in the system, yielding an implantable material, such as a polyurethane membrane. The drying temperature may range from 40° C. to 80° C., for example, 60° C., and the drying time may range from 6 to 24 hours, for example, 8 hours. An inert gas, such as nitrogen, may also be used for protection during drying.

The polyurethane material synthesized via the pre-polymerization method has a molecular chain including the structure shown in formula III:

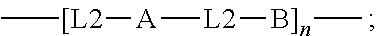

Wherein, L2 represents the first main chain unit (characteristics of the raw materials or chain units may be referenced from the preceding text; in this context, it denotes the first isocyanate), A represents the soft segment; and at least one of L2 and B is grafted with a functional side chain; and
A is independently selected from at least one of the following: polyether diol, polycarbonate diol, polyester diol, and polydimethylsiloxane diol.

The structural formula of the aforementioned B is:

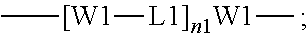

Where n1 is a natural number from 0 to 5;
L1 is an isocyanate (as defined above, i.e., the second isocyanate), such as at least one of the following: TDI, HDI, MDI, NDI, PPDI, IPDI, and XDI, wherein the hydrogen atom at each alkyl group in the isocyanate may be replaced by a functional side chain;
W1 is a chain extender (as defined above, i.e., the second main chain unit), which can be independently selected from at least one of the following: ethylene glycol, butanediol, hexanediol, octanediol, and ethylenediamine, and the hydrogen on each hydrocarbon group in X1 may be replaced by a functional side chain; and
at least one of L1 and W1 is partially grafted with a functional side chain.

The present disclosure provides several applications of implantable materials in the field of prosthetic heart valve materials.

The implantable material is applied to a prosthetic heart valve through processing. For example, the prosthetic heart valve provided in Example 1 includes a stent and valve leaflets connected to the stent. The leaflets are multiple pieces that cooperate with each other to control the opening and closing of the blood flow channel. The valve leaflets are made of the implantable material.

In the field of prosthetic heart valves, especially aortic valves, extremely stringent requirements exist for the mechanical properties of valve leaflets. These include tensile strength, elongation, elastic modulus, surface silicon content, surface phosphorylcholine group content, and surface sulfonic acid group content. Among these, higher silicon content, surface phosphorylcholine group content, and surface sulfonic acid group content generally indicate better biocompatibility, while also providing a degree of protection to the internal structure of the leaflet, thereby contributing to relatively superior biostability. The valve leaflets made of the implantable material of this disclosure are particularly suitable for aortic valves. During the preparation process, an annealing treatment may also be performed. The annealing conditions involve maintaining a temperature of 60° C. to 150° C. for 2 hours to 60 hours, which enables sufficient rearrangement of the internal molecular chains of the polyurethane, allowing the hard segments to arrange in a manner of minimum energy or close to minimum energy.

The above-mentioned implantable materials may also be used to fabricate a covering membrane, which is attached to the inner and/or outer sides of the stent to maintain sealing, prevent leakage and provide safety protection. The covering membrane on the inner side of the stent and the valve leaflets can be either separately connected or integrally connected as one piece, while the covering membrane on the outer side of the stent may be integrally formed by folding the inner covering membrane.

The following provides multiple specific examples with specific parameters. The raw materials used in each preparation example and embodiment may undergo a drying pretreatment according to moisture or impurity requirements before use, for example, vacuum drying at 80° C. for 10 hours.

### Preparation Example 1

Triphenylmethane triisocyanate (CAS: 2422-91-5) was placed in a three-necked flask. Under a nitrogen atmosphere and at 60° C., an equimolar amount of 3-((tert-butyldimethylsilyl)oxy)-propanol (CAS: 73842-99-6) was added dropwise using a dropping funnel. After the addition was completed, the mixture was reacted for 2 hours to obtain intermediate 1, i.e., isocyanate grafted with polysiloxane side chains as shown below:

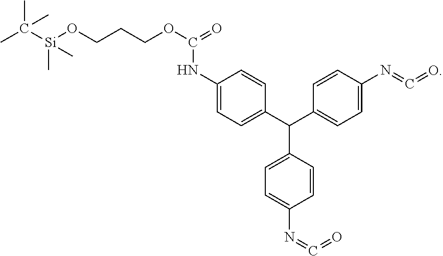 [SMILES: CC(C)(C)[Si](C)(C)OCCCOC(=O)Nc1ccc(C(c2ccc(N=C=O)cc2)c2ccc(N=C=O)cc2)cc1]

### Preparation Example 2

Triphenylmethane triisocyanate was placed in a three-necked flask. Under nitrogen atmosphere and at 60° C., an equimolar amount of isethionic acid (CAS: 107-36-8) was added dropwise into the flask using a dropping funnel. After the addition was completed, the mixture was reacted for 2 hours to obtain intermediate 2, i.e., isocyanate grafted with sulfonic acid side chains as shown below:

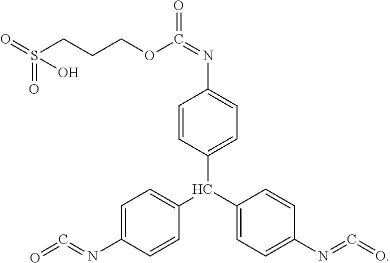

### Preparation Example 3

10.50 g of 2-amino-2-methyl-1,3-propanediol (AMPD, CAS 115-69-5) was dissolved in a mixture of 50 mL of 1,4-dioxane and 50 mL of water by stirring, and the dissolution was carried out in an ice-water bath. 25.92 g of di-tert-butyl dicarbonate ((Boc)₂O, CAS 24424-99-5) was added and stirred at room temperature for 24 hours. After stirring, the solution was concentrated under vacuo to yield an oily residue, which was then recrystallized to obtain the product BocAMP. 18.8 g of BocAMP and 25.4 mL of triethylamine were dissolved in 200 mL of anhydrous THF and cooled to 0° C. Under a nitrogen atmosphere, 14.5 mL of acetyl chloride was added dropwise to the system, and the mixture was then heated to room temperature and stirred for 2 hours. After the reaction was complete, the mixture was filtered and the THF was removed by rotary evaporation. The crude BDAPA was purified by silica gel column chromatography using ethyl acetate (EtOAc)/petroleum ether (1:3) to obtain the product BDAPA.

9.4 g of BDAPA was dissolved in 50 mL of ethyl acetate saturated with hydrogen chloride. The mixture was left to stand at room temperature for 2 hours, and then concentrated under reduced pressure to obtain DAPA. The resulting DAPA, along with 4.6 mL of triethylamine and 5.2 g of HDA, was dissolved in a mixture of 50 mL of dimethylformamide and 200 mL of tetrahydrofuran to obtain HDADEA.

4.5 g of HDADEA and 1.73 mL of triethylamine were dissolved in a mixture of 100 mL of anhydrous ether and 30 mL of anhydrous acetonitrile. After cooling to 20° C., 1.78 g of 2-chloro-1,3,2-dioxaphosphole-2-oxide (COP, CAS 6609-64-9) dissolved in 10 mL of anhydrous ether was slowly added to the stirred solution over 20 minutes under a nitrogen atmosphere. The reaction system was kept at 20° C. for 1 hour and then slowly warmed to room temperature. After the reaction, the triethylammonium chloride precipitate was filtered and washed with ether. The filtrate was evaporated in vacuo under a stream of nitrogen to yield a colorless oily residue. The residue was dissolved in 60 mL of anhydrous acetone nitrile and transferred to a 100 mL pressure bottle. After cooling the pressure bottle to 18° C., an excess of trimethylamine was quickly added to the solution. The pressure bottle was then sealed and maintained at 55° C. for 16 hours. The solution was then evaporated under vacuo to produce a viscous liquid. The viscous liquid was dissolved in methanol and an excess of ammonia was added. The mixture was stirred at room temperature for 24 hours and then concentrated under reduced pressure to obtain (9-(2-hydroxy-1-hydroxymethyl-1-methyl-ethylcarbamoyl)-nonylphosphatidylcholine) as shown below:

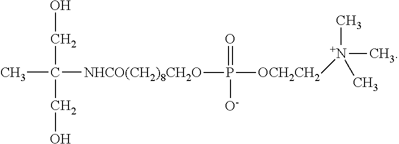

### Example 1

The synthesis method for implantable material specifically includes:
(1) Pre-polymering and end-capping the soft segment raw material using the first isocyanate, with MDI used as the first isocyanate, and polydimethylsiloxane diol used as the soft segment raw material.

Based on the mass percentage of the reaction system (excluding the solvent DMAc), 30 wt. % MDI was placed in a three-necked flask. At 80° C., 55 wt. % polydimethylsiloxane diol (molecular weight: 1000) was added dropwise into the three-necked flask using a dropping funnel. After the addition was completed, the reaction was allowed to proceed for 2 hours to produce a prepolymer, MDI-PDMS-MDI.
(2) Cooling the system after the reaction in step (1) to 50° C. and adding 9 wt. % of the second isocyanate to it, with the intermediate 1 of Example 1 serving as the second isocyanate, and pre-dissolving the second isocyanate in DMAc to form a 20 (w/v) % solution, and stirring it for 10 to 30 minutes.
(3) Raising the temperature of the solution from step (2) to 80° C., adding dropwise 6 wt. % of chain extender BDO to the solution, and carrying out a chain extension reaction for 2 hours after the addition is completed.
(4) Drying the solution at 60° C. to obtain a polyurethane membrane. The synthesis process was carried out under a nitrogen atmosphere. Furthermore, the valve leaflets can be prepared using the polyurethane membrane and annealed at 150° C. for 2 hours.

### Example 2

The synthesis method for implantable material specifically includes:
(1) Pre-polymering and end-capping the soft segment raw material using the first isocyanate, with MDI used as the first isocyanate, and polydimethylsiloxane diol and polyhexanediol (PHMO) used as soft segment raw materials.

Based on the mass percentage of the reaction system (excluding the solvent DMAc), 30 wt. % MDI was placed in a three-necked flask. At 80° C., a mixture of 44 wt. % polydimethylsiloxane diol (molecular weight, 1000) and 11 wt. % PHMO (molecular weight, 700) was added dropwise using a dropping funnel. After the addition was completed, the mixture was reacted for 2 hours to produce prepolymers 1, MDI-PDMS-MDI and MDI-PHMO-MDI.
(2) Cooling the system after the reaction in step (1) to 50° C. and adding 9 wt. % of the second isocyanate to it, with the intermediate 1 of Example 1 serving as the second isocyanate, and pre-dissolving the second isocyanate in DMAc to form a 20 (w/v) % solution, and stir it for 10 to 30 minutes.
(3) Raising the temperature of the solution from step (2) to 80° C., adding dropwise 6 wt. % of chain extender BDO to the solution, and carrying out chain extension reaction for 2 hours after the addition is completed.
(4) Drying the solution at 60° C. to obtain a polyurethane membrane.

The synthesis process was carried out under a nitrogen atmosphere. Furthermore, the valve leaflets can be prepared using polyurethane membrane and annealed at 150° C. for 2 hours.

### Example 3

The synthesis method for implantable material specifically includes:
(1) Pre-polymering and end-capping the soft segment raw material using the first isocyanate, with MDI used as the first isocyanate, and polydimethylsiloxane diol used as the soft segment raw material.

Based on the mass percentage of the reaction system (excluding the solvent DMAc), 33.0 wt. % MDI was placed in a three-necked flask. At 80° C., 54.0 wt. % polydimethylsiloxane diol (molecular weight: 1000) was added dropwise into the three-necked flask using a dropping funnel. After the addition was completed, the reaction was allowed to proceed for 2 hours to produce a prepolymer, MDI-PDMS-MDI.
(2) Adding 7.0 wt. % 9-(2-hydroxy-1-hydroxymethyl-1-methyl-ethylcarbamoyl)-nonylphosphatidylcholine dropwise to the system of step (1), and performing the chain extension reaction for 2 hours after the addition is completed.
(3) Adding DMAc to form a 20 (w/v) % solution.
(4) Adding 6.0 wt. % of chain extender BDO dropwise to the solution from step (3) at 80° C., and performing the chain extension reaction for 2 hours after the addition is complete.
(5) Drying the polyurethane membrane at 60° C. after the chain extension reaction is completed, to obtain a polyurethane membrane.

The synthesis process was carried out under a nitrogen atmosphere. Furthermore, the valve leaflets can be prepared using polyurethane membrane and annealed at 150° C. for 2 hours.

### Example 4

The synthesis method for implantable material specifically includes:
(1) Pre-polymering the soft segment raw material and end-capping it using the first isocyanate, with MDI used as the first isocyanate, and polydimethylsiloxane diol and polyhexanediol (PHMO) used as soft segment raw materials.

Based on the mass percentage of the reaction system (excluding the solvent DMAc), 33 wt. % MDI was placed in a three-necked flask. At 80° C., a mixture of 43.2 wt. % polydimethylsiloxane diol (molecular weight, 1000) and 10.8 wt. % PHMO (molecular weight, 700) was added dropwise using a dropping funnel. After the addition was completed, the mixture was reacted for 2 hours to produce prepolymers, MDI-PDMS-MDI and MDI-PHMO-MDI.
(2) Adding 7.0 wt. % 9-(2-hydroxy-1-hydroxymethyl-1-methyl-ethylcarbamoyl)-nonylphosphatidylcholine added dropwise at 80° C., and performing a chain extension reaction for 2 hours after the addition is completed.
(3) Adding DMAc to form a 20 (w/v) % solution.
(4) Adding 6.0 wt. % of chain extender BDO dropwise to the solution from step (3) and performing the chain extension reaction for 2 hours after the addition is complete.
(5) Drying the solution at 60° C. to obtain a polyurethane membrane.

The synthesis process was carried out under a nitrogen atmosphere. Furthermore, the valve leaflets can be prepared using the polyurethane membrane and annealed at 150° C. for 2 hours.

### Example 5

The synthesis method for implantable material specifically includes:
(1) Pre-polymering the soft segment raw material and end-capping it using the first isocyanate, with MDI used as the first isocyanate, and polydimethylsiloxane diol and polyhexanediol (PHMO) used as soft segment raw materials.

Based on the mass percentage of the reaction system (excluding the solvent DMAc), 32 wt. % MDI was placed in a three-necked flask. At 80° C., a mixture of 40 wt. % polydimethylsiloxane diol (molecular weight, 1000) and 10 wt. % PHMO (molecular weight, 700) was added dropwise using a dropping funnel. After the addition was completed, the mixture was reacted for 2 hours to produce prepolymers 1, MI-PDMS-MDI and MDI-PHMO-MDI.
(2) Cooling the system after the reaction in step (1) to 50° C. and adding 8 wt. % of the second isocyanate to it, with the intermediate 1 of Example 1 serving as the second isocyanate, and pre-dissolving the second isocyanate in DMAc to form a 20 (w/v) % solution, and stirring it for 10 to 30 minutes.
(3) Adding 5 wt. % of the chain extender 9-(2-hydroxy-1-hydroxymethyl-1-methyl-ethylcarbamoyl)-nonylphosphatidylcholine dropwise to the solution from step (2) at 80° C., and performing the chain extension reaction for 2 hours after the addition is complete.
(4) Adding 5 wt. % of the chain extender BDO dropwise to the reaction system from step (3) at 80° C., and performing the chain extension reaction for 2 hours after the addition is complete.
(5) Drying the solution at 60° C. to obtain a polyurethane membrane.

The synthesis process was carried out under a nitrogen atmosphere. Furthermore, the valve leaflets can be prepared using the polyurethane membrane and annealed at 150° C. for 2 hours.

### Example 6

The synthesis method for implantable material specifically includes:
(1) Pre-polymering the soft segment raw material and end-capping it using the first isocyanate, with MDI used as the first isocyanate, and polydimethylsiloxane diol and polyhexanediol (PHMO) used as soft segment raw materials.

Based on the mass percentage of the reaction system (excluding the solvent DMAc), 33 wt. % MDI was placed in a three-necked flask. At 80° C., a mixture of 44 wt. % polydimethylsiloxane diol (molecular weight, 1000) and 11 wt. % PHMO (molecular weight, 700) was added dropwise using a dropping funnel. After the addition was completed, the mixture was reacted for 2 hours to produce prepolymers 1, MI-PDMS-MDI and MDI-PHMO-MDI.
(2) Cooling the system after the reaction in step (1) to 50° C. and adding 6 wt. % of the second isocyanate to it, with the intermediate 2 of Example 2 serving as the second isocyanate, and pre-dissolving the second isocyanate in DMAc to form a 20 (w/v) % solution, and stirring it for 10 minutes to 30 minutes.
(3) Raising the temperature of the solution from step (2) to 80° C., adding dropwise 6 wt. % of chain extender BDO to the solution, and performing the chain extension reaction for 2 hours after the addition is completed.
(4) Drying the solution at 60° C. to obtain a polyurethane membrane.

The synthesis process was carried out under a nitrogen atmosphere. Furthermore, the valve leaflets can be prepared using polyurethane membrane and annealed at 150° C. for 2 hours.

### Example 7

The synthesis method for implantable material specifically includes:
(1) Adding 30 wt. % MDI, 44 wt. % polydimethylsiloxane diol (molecular weight of 1000), 11 wt. % PHMO (molecular weight of 700), 9 wt. % intermediate 1, and 6 wt. % chain extender BDO to a three-necked flask according to the mass percentage of the reaction system (excluding the solvent DMAc), then adding DMAc to form a 20 (w/v) % solution, and performing a reaction at 80° C. for 4 h.
(2) Drying the solution at 60° C. to obtain a polyurethane membrane.

The synthesis process was carried out under a nitrogen atmosphere. Furthermore, the valve leaflets can be prepared using the polyurethane membrane and annealed at 150° C. for 2 hours.

### Example 8

The synthesis method for implantable material specifically includes:
(1) Adding 33 wt. % MDI, 43.2 wt. % polydimethylsiloxane diol (molecular weight: 1000), 10.8 wt. % PHMO (molecular weight: 700), and 7.0 wt. % 9-(2-hydroxy-1-hydroxymethyl-1-methyl-ethylcarbamoyl)-nonylphosphatidylcholine, and 6 wt. % BDO to a three-necked flask according to the mass percentage of the reaction system (excluding the solvent DMAc), then adding DMAc to form a 20 (w/v) % solution, and performing a reaction at 80° C. for 4 h under nitrogen atmosphere.
(2) Drying the solution at 60° C. to obtain a polyurethane membrane.

The synthesis process was carried out under a nitrogen atmosphere. Furthermore, the valve leaflets can be prepared using the polyurethane membrane and annealed at 150° C. for 2 hours.

### Control Example 1

(1) Based on the mass percentage of the reaction system (excluding the solvent DMAc), 39 wt. % MDI was placed in a three-necked flask. Under a nitrogen atmosphere and at 80° C., 55 wt. % polydimethylsiloxane diol with a molecular weight of 1000 was added dropwise to the three-necked flask. After the dropwise addition was completed, the reaction was allowed to proceed for 2 hours to obtain an intermediate 1, the prepolymer 1: MI-PDMS-MDI.
(2) At 80° C., 6.0 wt. % of the chain extender BDO was added dropwise. After the addition was complete, the chain extension reaction was carried out for 2 hours.
(3) DMAc was added to form a 20 (w/v) % solution.
(2) Drying was performed at 60° C. to obtain a polyurethane membrane.

The synthesis process was carried out under a nitrogen atmosphere. Furthermore, the valve leaflets can be prepared using the polyurethane membrane and annealed at 150° C. for 2 hours.

### Control Example 2

(1) Based on the mass percentage of the reaction system (excluding the solvent DMAc), 39 wt. % of MDI was placed in a three-necked flask. Under a nitrogen atmosphere and at 80° C., 44 wt. % of polydimethylsiloxane diol with a molecular weight of 1000 and 11 wt. % of polyhexanediol (PHMO) with a molecular weight of 700 were added dropwise to the three-necked flask. After the addition was completed, the mixture was allowed to react for 2 hours to obtain intermediates 1, the prepolymers 1: MI-PDMS-MDI and MDI-PHMO-MDI.
(2) At 80 C, 6.0 wt. % of the chain extender BDO was added dropwise. After the addition was complete, the chain extension reaction was carried out for 2 hours.
(3) DMAc was added to form a 20 (w/v) % solution.
(2) Drying was performed at 60° C. to obtain a polyurethane membrane.

The synthesis process was carried out under a nitrogen atmosphere. Furthermore, the valve leaflets can be prepared using the polyurethane membrane and annealed at 150° C. for 2 hours.

### Control Example 3

Commercially available Elast-Eon 2™ 85A polyurethane material.

The raw materials used in each example and control example are shown in Table 1.

TABLE 1
Raw materials used in each example and control example

| | First | Second | Soft segment | Chain |
|---|---|---|---|---|
| Group | isocyanate | isocyanate | raw materials | Extender |
| Example 1 | MDI | Intermediate 1 | Polydimethylsiloxane | BDO |
| | | | diol | |
| Example 2 | MDI | Intermediate 1 | Polydimethylsiloxane | BDO |
| | | | diol | |
| | | | Polyhexanediol | |
| Example 3 | MDI | — | Polydimethylsiloxane | BDO |
| | | | diol | 9-(2-Hydroxy-1- |
| | | | | hydroxymethyl-1- |
| | | | | methyl- |
| | | | | ethylcarbamoyl)- |
| | | | | nonylphosphatidylcholine |
| Example 4 | MDI | — | Polydimethylsiloxane | BDO |
| | | | diol | |
| | | | Polyhexanediol | 9-(2-Hydroxy-1- |
| | | | | hydroxymethyl-1- |
| | | | | methyl- |
| | | | | ethylcarbamoyl)- |
| | | | | nonylphosphatidylcholine |
| Example 5 | MDI | Intermediate 1 | Polydimethylsiloxane | BDO |
| | | | diol | |
| | | | Polyhexanediol | 9-(2-Hydroxy-1- |
| | | | | hydroxymethyl-1- |
| | | | | methyl- |
| | | | | ethylcarbamoyl)- |
| | | | | nonylphosphatidylcholine |
| Example 6 | MDI | Intermediate 2 | Polydimethylsiloxane | BDO |
| Sulfonic | | | diol | |
| acid group | | | Polyhexanediol | |
| Example 7 | MDI | Intermediate 1 | Polydimethylsiloxane | BDO |
| | | | diol | |
| | | | Polyhexanediol | |
| Example 8 | MDI | — | Polydimethylsiloxane | BDO |
| | | | diol | |
| | | | Polyhexanediol | 9-(2-Hydroxy-1- |
| | | | | hydroxymethyl-1- |
| | | | | methyl- |
| | | | | ethylcarbamoyl)- |
| | | | | nonylphosphatidylcholine |
| Control | MDI | — | Polydimethylsiloxane | BDO |
| Example 1 | | | diol | |
| Control | MDI | — | Polydimethylsiloxane | BDO |
| Example 2 | | | diol | |
| | | | Polyhexanediol | |
| Control | Elast-Eon 2 ™ polyurethane material | | | |
| Example 3 | | | | |

The amounts of raw materials used in the examples and control examples are shown in Table 2.

TABLE 2
Amounts of raw materials used in various
examples and control examples

| | First | Second | Soft segment | Chain |
|---|---|---|---|---|
| Group | isocyanate | isocyanate | raw materials | Extender |
| Example 1 | 30 | 9 | 55 | 6 |
| Example 2 | 30 | 9 | 44 | 6 |
| | | | 11 | |
| Example 3 | 33 | — | 43.2 | 6 |
| | | | 10.8 | 7 |
| Example 4 | 33 | — | 54.0 | 6 |
| | | | | 7 |
| Example 5 | 32 | 8 | 40 | 5 |
| | | | 10 | 5 |
| Example 6 | 33 | 6 | 44 | 6 |
| | | | 11 | |
| Example 7 | 30 | 9 | 44 | 6 |
| | | | 11 | |
| Example 8 | 33 | — | 43.2 | 6 |
| | | | 10.8 | 7 |
| Control | 39 | — | 55 | 6 |
| Example 1 | | | | |
| Control | 39 | — | 44 | 6 |
| Example 2 | | | 11 | |

Examples 1 to 6 all employed the pre-polymerization method to prepare the implantable materials, wherein:

In Example 1, MDI was an isocyanate not grafted with functional side chains, and intermediate 1 was an isocyanate grafted with functional side chains (dimethylsiloxane groups). The two respectively form independent first main chain units, with a molar ratio of 7:1. Based on this molar ratio, it was calculated that the first main chain units grafted with functional side chains account for 12.50% of the total molar amount of all first main chain units.

In Example 2, the molar ratio of polydimethylsiloxane diol to polyhexanediol was 4:1. The molar ratio of MDI to intermediate 1 was 7:1. Based on this molar ratio, the first main chain units grafted with functional side chains account for 12.50% of the total molar amount of all first main chain units.

In Example 3, BDO was a chain extender not grafted with functional side chains, while 9-(2-hydroxy-1-hydroxymethyl-1-methyl-ethylcarbamoyl)-nonylphosphatidylcholine was a chain extender grafted with functional side chains (phosphatidylcholine groups). The two independently form independent second main chain units, with a molar ratio of 4.2:1. Based on this molar ratio, the second main chain units grafted with functional side chains account for 19.23% of the total molar amount of all second main chain units.

In Example 4, the molar ratio of polydimethylsiloxane diol to polyethylene glycol was 4:1. BDO was a chain extender without grafted functional side chains, and 9-(2-hydroxy-1-hydroxymethyl-1-methyl-ethylcarbamoyl)-nonylphosphatidylcholine was a chain extender grafted with functional side chains (phosphatidylcholine groups). The two independently formed second main chain units, and the molar ratio was 4.2:1. Based on this molar ratio, the second main chain units grafted with functional side chains accounted for 19.23% of the total molar amount of all second main chain units.

In Example 5, MDI was an isocyanate without grafted functional side chains, and intermediate 1 was an isocyanate with grafted functional side chains. The two respectively form independent first main chain units, and the molar ratio was 8.9:1; BDO was a chain extender without grafted functional side chains, and 9-(2-hydroxy-1-hydroxymethyl-1-methyl-ethylcarbamoyl)-nonylphosphatidylcholine was a chain extender grafted with functional side chains. The two respectively form independent second main chain units, with a molar ratio of 4.9:1. Based on the two molar ratios, the first main chain units and the second main chain units grafted with functional side chains account for 12.66% of the total molar amount of all first main chain units and second main chain units.

In Example 6, MDI was an isocyanate without grafted functional side chains, and intermediate 2 was an isocyanate grafted with functional side chains (sulfonic acid groups). The molar ratio of the two was 10.85:1. Based on the molar ratio, the first main chain units grafted with functional side chains account for 8.4% of the total molar amount of all first main chain units.

Examples 7 and 8 employed a one-step synthesis method to prepare implantable materials, wherein:

In Example 7, MDI was an isocyanate without grafted functional side chains, and intermediate 1 was an isocyanate grafted with functional side chains. The two form independent first main chain units, with a molar ratio of 7:1. The molar ratio was calculated to show that the first main chain units grafted with functional side chains account for 12.50% of the total molar amount of all first main chain units.

In Example 8, BDO served as a chain extender without grafted functional side chains, and 9-(2-hydroxy-1-hydroxymethyl-1-methyl-ethylcarbamoyl)-nonylphosphatidylcholine served as a chain extender grafted with functional side chains. The two independently formed second main chain units, with a molar ratio of 4.2:1. Based on the molar ratio, the second main chain units grafted with functional side chains accounted for 19.2300 of the total molar amount of all second main chain units.

The tensile strength, elongation, elastic modulus and surface silicon content of the leaflets prepared in each embodiment and control example were tested. The test results are shown in Table 3.

TABLE 3
Performance test results

| | Tensile | | Elastic | Surface | Surface | Surface |
|---|---|---|---|---|---|---|
| | strength | Elongation | modulus | silicon | phosphorus | sulfur |
| Group | (MPa) | (%) | (MPa) | content (%) | content (%) | content (%) |
| Example | 24.3 | 520 | 32 | 70 | — | — |
| 1 | | | | | | |
| Example | 45.1 | 821 | 30 | 63 | — | — |
| 2 | | | | | | |
| Example | 23.1 | 520 | 32 | 37 | 26 | — |
| 3 | | | | | | |
| Example | 44.7 | 760 | 31 | 20 | 25 | — |
| 4 | | | | | | |
| Example | 43.5 | 750 | 29 | 57 | 10 | — |
| 5 | | | | | | |
| Example | 44.3 | 720 | 31 | 24 | — | 10 |
| 6 | | | | | | |
| Example | 46.3 | 600 | 51 | 68 | — | — |
| 7 | | | | | | |
| Example | 45.2 | 620 | 52 | 16 | 30 | — |
| 8 | | | | | | |
| Control | 15.2 | 370 | 32 | 44 | — | — |
| Example | | | | | | |
| 1 | | | | | | |
| Control | 38.3 | 760 | 33 | 37 | — | — |
| Example | | | | | | |
| 2 | | | | | | |
| Control | 28 | 580 | 33 | 20 | — | — |
| Example | | | | | | |
| 3 | | | | | | |

### Result Analysis

(1) Effect of Side Chains:

Based on the application scenario of the valve leaflet, it is required to possess suitable softness and strength. The softness is generally characterized by the elastic modulus, while the strength includes the tensile strength. Typically, under the condition that the elastic modulus is basically the same, a higher tensile strength indicates superior mechanical properties of the valve leaflet.

As shown in Table 3, compared to Control Example 1, the results of Examples 1 to 6 demonstrate that grafting functional side chains with suitable solubility parameters onto the hard segments can enhance the tensile strength of the valve leaflet. Furthermore, from the perspective of elongation, the valve leaflets from Examples 1 to 6 all exhibited elongation greater than 500%, meeting the application requirements for the valve leaflet.

### (2) Effect of the Second Soft Segment:

Comparing to Example 1, polyhexanediol added in Example 2 served as the second soft segment mentioned above. Since the second soft segment plays a transitional role between the first soft segment and the hard segment, it helps to further promote stress transfer between the hard and soft segments, reducing the likelihood of stress accumulation. This ultimately manifests as an improvement in the mechanical properties of the valve leaflet. Referring to Table 3, the tensile strength and elongation of Example 2 are significantly improved, indicating that stress can be transferred more smoothly.

Similarly, comparing Example 4 with Example 3, the tensile strength and elongation of Example 4 are significantly improved, which indicates that stress transfer proceeds relatively smoothly.

### (3) Biocompatibility and Stability:

Compared with Control Example 1, the introduction of polysiloxane side chains significantly increases the surface silicon content of the valve leaflets of Examples 1 and 2. Generally, a higher silicon content leads to better biocompatibility and stability of the valve leaflet.

In examples 3, 4, and 5, the BDO was partially replaced by the chain extender grafted with phosphorylcholine side chains. The phosphorylcholine on the hard segment was miscible in PDMS, thereby increasing the compatibility between the hard and soft segments. This allowed stress to transfer between the hard and soft segments, reducing local stress concentration. As a result, the tensile strength was significantly improved. Furthermore, since phosphorylcholine was positioned in the side chains, it experienced significant steric hindrance with surrounding groups, forcing it to migrate toward the surface. Consequently, the surface silicon content showed a slight decrease. However, the introduction of phosphorylcholine also altered the distribution of surface groups. As phosphorylcholine is a group with excellent biocompatibility and biostability, its tendency to migrate to the surface can further enhance the biostability and biocompatibility of the membrane.

Example 6 employed an isocyanate with sulfonic acid side chains. The sulfonic acid groups on the hard segments were miscible with the soft segments, thereby enhancing compatibility between hard and soft segments. Stress can be transferred between the hard and soft segments, reducing the concentration of local stress. As a result, the tensile strength was significantly improved. Furthermore, since the sulfonic acid groups were positioned in the side chains, they experienced significant steric hindrance with surrounding groups, forcing them to migrate toward the surface. Consequently, the surface silicon content showed a slight decrease. However, the introduction of sulfonic acid groups also altered the distribution of surface groups, resulting in fewer exposed polyether groups on the surface. This protects the groups most susceptible to oxidative cleavage, thereby enhancing the biostability of polyurethane materials.

The technical features of the above embodiments can be arbitrarily combined, and not all possible combinations of the technical features of the above embodiments have been described for the sake of brevity of description. However, as long as there is no contradiction in the combination of these technical characteristics, such combination should be regarded as falling into the scope of this specification. When technical features of different embodiments are embodied in the same drawing, it can be deemed that the drawing also discloses examples of combinations of the various embodiments involved.

The above-described embodiments only illustrate several embodiments of the present disclosure, and the descriptions thereof are specific and detail, but should not be construed as limiting the scope of the patent disclosure. It should be noted that, for those of ordinary skill in the art, several modifications and improvements can be made without departing from the concept of the present disclosure, all of which fall into the protection scope of the present disclosure.

What is claimed is:

1. An implantable material made of polyurethane, wherein a molecular chain structure of the polyurethane comprises a soft segment and a hard segment, the hard segment is grafted with a functional side chain which has a solubility parameter in a range of 6 to 8, and a raw material for the soft segment is an oligomer diol.

2. The implantable material according to claim 1, wherein the functional side chain is at least one selected from the following: a dimethylsiloxane group, a polydimethylsiloxane group, a phosphorylcholine group, and a sulfonic acid group, and the functional side chain has a molecular weight in a range of 100 to 2000.

3. The implantable material according to claim 1, wherein the oligomer diol is at least one selected from the following: polycarbonate diol, polyester diol, polyether diol, and polydimethylsiloxane diol; wherein the polycarbonate diol is at least one of Duranol T5651 and Duranol T5652, the polyester diol is at least one of adipic acid polyester diol and succinic acid polyester diol, and the polyether diol is at least one of polyhexanediol and polytetrahydrofuran diol.

4. The implantable material according to claim 1, wherein the soft segment comprises a first soft segment and a second soft segment;
wherein a raw material for the first soft segment has a molecular weight in a range of 500 to 2000, with a solubility parameter in a range of 6 to 7;
wherein a raw material for the second soft segment has a molecular weight in a range of 500 to 2000, with a solubility parameter in a range of 8 to 10; and
a mass ratio of the first soft segment to the second soft segment in the polyurethane is in a range of 2.5:1 to 5:1.

5. The implantable material according to claim 1, wherein the hard segment comprises:
a first main chain unit derived from isocyanate; and
a second main chain unit derived from a chain extender,
wherein the functional side chain is grafted to at least one of the first main chain unit and the second main chain unit.

6. The implantable material according to claim 5, wherein a raw material for the first main chain unit is at least one of the following: toluene diisocyanate, hexamethylene diisocyanate, diphenylmethane diisocyanate, naphthalene diisocyanate, p-phenylene diisocyanate, isophorone diisocyanate, xylylene diisocyanate, and triphenylmethane triisocyanate;
wherein a raw material for the second main chain unit is selected from at least one of small molecule diol and small molecule diamine;
wherein the small molecule diol is at least one of the following: ethylene glycol, butanediol (BDO), hexanediol, and octanediol; and
wherein the small molecule diamine is at least one of ethylenediamine, butanediamine, and hexamethylenediamine.

7. The implantable material according to claim 5, wherein the functional side chain is grafted in one of following three ways:
only onto the first main chain unit;
only onto the second main chain unit; and
onto both the first main chain unit and the second main chain unit.

8. The implantable material according to claim 7, wherein in case the functional side chain is grafted:
only onto the first main chain unit, the first main chain unit grafted with the functional side chain accounts for 5% to 20% of a total molar amount of all first main chain units; or
only onto the second main chain unit, the second main chain unit grafted with the functional side chain accounts for 5% to 20% of the total molar amount of all second main chain units; or
onto both the first main chain unit and the second main chain unit, the first main chain unit and the second main chain unit grafted with the functional side chain account for 5% to 20% of the total molar amount of all the first main chain units and the second main chain units.

9. The implantable material according to claim 1, wherein the polyurethane has an isocyanate index (R) in a range of 1.0 to 1.1, or
the hard segment has a solubility parameter in a range of 11 to 14; or
the hard segment accounts for 35% to 55% of a total mass of the polyurethane.

10. A synthesis method for an implantable material according to claim 1, comprising:
providing a soft segment raw material and a hard segment raw material; and
reacting the soft segment raw material with the hard segment raw material to obtain the implantable material,
wherein the hard segment raw material comprises isocyanate and a chain extender, wherein at least one of the isocyanate and the chain extender is grafted with a functional side chain, and the functional side chain has a solubility parameter in a range of 6 to 8.

11. The synthesis method according to claim 10, wherein the functional side chain is at least one of the following: a dimethylsiloxane group, a polydimethylsiloxane group, a phosphorylcholine group, and a sulfonic acid group;
the functional side chain has a molecular weight in a range of 100 to 2000; and
the functional side chain is pre-grafted onto the isocyanate and/or the chain extender serving as the hard segment raw material; or
the functional side chain is grafted during reaction process.

12. The synthesis method according to claim 10, comprising:
S100, pre-polymerizing and capping the soft segment raw material with the isocyanate to obtain a prepolymer; and
S200, reacting the prepolymer with a chain extender to obtain the implantable material,
wherein the isocyanate in step S100 is a first isocyanate, and a second isocyanate is further added in step S200, wherein the first isocyanate and the second isocyanate are each a single isocyanate or a mixture of a plurality of isocyanates.

13. The synthesis method according to claim 12, wherein a composition of the mixture of the plurality of isocyanates is one of the following:
a. isocyanates all not grafted with the functional side chain;
b. isocyanates all grafted with the functional side chain; and
c. a combination of isocyanates grafted with the functional side chain and isocyanates not grafted with the functional side chain.

14. The synthesis method according to claim 13, wherein in the mixture of the plurality of isocyanates, a molar ratio of the isocyanate not grafted with the functional side chain to the isocyanate grafted with the functional side chain is in a range of 4:1 to 20:1, and the isocyanate not grafted with the functional side chain is at least one of the following: toluene diisocyanate, hexamethylene diisocyanate, diphenylmethane diisocyanate, naphthalene diisocyanate, p-phenylene diisocyanate, isophorone diisocyanate, xylylene diisocyanate, and triphenylmethane triisocyanate.

15. The synthesis method according to claim 12, wherein the soft segment raw material in step S100 comprises a first soft segment raw material and a second soft segment raw material, and the first soft segment raw material and the second soft segment raw material are both pre-polymerized and end-capped with the isocyanate;
wherein the first soft segment raw material has a molecular weight in a range of 500 to 2000, with a solubility parameter in a range of 6 to 7; and
wherein the second soft segment raw material has a molecular weight in a range of 500 to 2000, with a solubility parameter in a range of 8 to 10.

16. The synthesis method according to claim 15, wherein the first soft segment raw material is polydimethylsiloxane diol, the second soft segment raw material is at least one of polyether diol, polycarbonate diol, and polyester diol, and a mass ratio of the first soft segment raw material to the second soft segment raw material is in a range of 2.5:1 to 5:1; and
wherein in step S100, the first soft segment raw material and the second soft segment raw material are pre-mixed before being fed.

17. The synthesis method according to claim 12, wherein the chain extender comprises a linear chain extender and a chain extender grafted with a functional side chain;
wherein the linear chain extender is at least one of a small molecule diol and a small molecule diamine;
wherein a main chain of the chain extender grafted with the functional side chain is identical to that of the linear chain extender; and
wherein the chain extender grafted with the functional side chain is at least one of 1-palmitoylpropanediol-3-phosphocholine, 9-(2-hydroxy-1-hydroxymethyl-1-methyl-ethylcarbamoyl)-nonylphosphatidylcholine, and sphingosine phosphorylcholine.

18. The synthesis method according to claim 17, wherein a molar ratio of the linear chain extender to the chain extender grafted with the functional side chain is in a range of 4:1 to 20:1, a mixture of the linear chain extender and the chain extender grafted with the functional side chain is added in step S200, and step S200 comprises:
in a first reaction stage, adding the chain extender grafted with the functional side chain to react with the prepolymer, wherein a temperature of the first reaction stage is in a range of 60° C. to 80° C., with a duration of 1 hour to 2 hours; and
in a second reaction stage, adding the linear chain extender to continue to react after completion of the first reaction stage, wherein a temperature of the second reaction stage is in a range of 60° C. to 80° C., with a duration of 1 hour to 2 hours.

19. The synthesis method according to claim 12, wherein a chain extension reaction system further comprises a solvent, and the solvent is at least one of the following: N,N-dimethylacetamide, N,N-dimethylformamide, tetrahydrofuran, dimethyl sulfoxide, and toluene.

20. A prosthetic heart valve comprising a stent and valve leaflets connected to the stent, wherein the valve leaflets are made of the implantable material according to claim 1, and the valve leaflets comprise a plurality of pieces that cooperate with each other to control opening and closing of a blood flow channel.
